(19) United States
(12) Patent Application Publication
(10) Pub. No.: US 2008/0054232 A1
(43) Pub. Date: Mar. 6, 2008

(54) PREPARATION OF HIGH ASSAY DECABROMODIPHENYL OXIDE

(75) Inventor: Bonnie Gary McKinnie, Magnolia, AR (US)

Correspondence Address: ALBEMARLE CORPORATION, 451 FLORIDA STREET, BATON ROUGE, LA, 70801-1765, US

(73) Assignee: ALBEMARLE CORPORATION, Baton Rouge, LA (US)

(21) Appl. No.: 11/843,734

(22) Filed: Aug. 23, 2007

Related U.S. Application Data
(60) Provisional application No. 60/823,811, filed on Aug. 29, 2006.

(51) Int. Cl. C09K 21/08; C07C 25/00

(52) U.S. Cl. 252/609; 570/201

(57) ABSTRACT

Process technology for producing very pure reaction-derived decabromodiphenyl oxide is described. Diphenyl oxide or partially brominated diphenyl oxide or a mixture of either or both of these is fed substantially continuously over a period of about 2 to about 12 hours into a reactor containing an excess of refluxing bromine containing Lewis acid bromination catalyst, and substantially concurrently reducing the content of hydrogen bromide present in the reactor whereby a decabromodiphenyl oxide product having a purity of over 99%, preferably 99.5% or more, is formed in the reactor.

### REFERENCE TO RELATED APPLICATION

This application claims the benefit and priority of U.S. Provisional Application No. 60/823,811, filed Aug. 29, 2006, the disclosure of which is incorporated herein by reference.

### TECHNICAL FIELD

This invention relates to the preparation of high assay decabromodiphenyl oxide products, and their use in flammable materials.

### BACKGROUND

Decabromodiphenyl oxide (DBDPO) is a time-proven flame retardant for use in many flammable macromolecular materials, e.g. thermoplastics, thermosets, cellulosic materials and back coating applications.

DBDPO is presently sold as a powder derived from the bromination of diphenyl oxide or a partially brominated DPO containing an average of about 0.7 bromine atom per molecule of DPO. Such bromination is conducted in excess bromine and in the presence of a bromination catalyst, usually AlCl₃. The operation is typically conducted at 177° F. (ca. 80.5° C.) with a 2-3 hour feed time. The powdered products are not 100% DBDPO, but rather are mixtures that contain up to about 98% DBDPO and about 1.5%, or a little more, of nonabromodiphenyl oxide co-product. As a partially brominated product, this amount of nonabromodiphenyl oxide is considered problematic by some environmental entities.

It would therefore be desirable to provide process technology enabling preparation of DBDPO products of higher purity, such as products comprising (i) more than 99% of DBDPO and (ii) nonabromodiphenyl oxide in an amount not exceeding 0.5%, preferably not exceeding 0.3%, and still more preferably, not exceeding about 0.1%. It would be especially desirable if such technology could produce DBDPO products comprising (i) at least 99.5% of DBDPO and (ii) nonabromodiphenyl oxide in an amount not exceeding 0.5%, preferably not exceeding 0.3%, and still more preferably, not exceeding about 0.1%.

### BRIEF DESCRIPTION OF THE DRAWINGS

FIG. 1 is a copy of a GC trace of the product of Example 4 hereinafter.

FIG. 2 is a copy of a GC trace of the product of Example 6 hereinafter.

### BRIEF SUMMARY OF THE INVENTION

It has now been found possible to directly produce without recourse to recrystallization or chromatographic purification steps DBDPO products having such higher amounts of DBDPO and lower contents of nonabromodiphenyl oxides. This can be accomplished by maintaining a substantially continuous, coordinated time-temperature feed of diphenyl oxide (DPO) and/or partially brominated DPO of feed to a reactor containing a refluxing reaction mixture comprising an excess of bromine in which Lewis acid bromination catalyst is present, and substantially concurrently reducing sufficiently the amount of hydrogen bromide coproduct present in the reactor so that a DBDPO product containing more than 99% of DBDPO is formed in the reactor.

The postulation upon which this invention was based is that when brominating DPO and/or partially brominated DPO with excess bromine in the presence of a Lewis acid catalyst, an equilibrium exists between nonabromodiphenyl oxide and decabromodiphenyl oxide which can be depicted as follows:

Br₉-DPO+Br₂ [symbol] Br₁₀-DPO+HBr

and that a prolonged feed of the DPO and/or partially brominated DPO to refluxing bromine while substantially concurrently reducing hydrogen bromide content in the reactor enables a shift to the right in this equilibrium so that the amount of nonabromodiphenyl oxide is diminished and more of the desired decabromodiphenyl oxide forms and precipitates with less nonabromodiphenyl oxide being coprecipitated within the decabromodiphenyl oxide particles. It is also believed that if the DPO and/or partially brominated DPO is fed too rapidly, the precipitation of at least one Br₉-DPO isomer occurs so rapidly that the above equilibrium is not totally reached.

Thus, in accordance with one of the embodiments of this invention, there is provided a process of preparing reaction-derived decabromodiphenyl oxide product of high purity, which process comprises feeding diphenyl oxide and/or partially brominated diphenyl oxide substantially continuously over a period in the range of about 2 to about 12 hours into a reactor containing a refluxing reaction mixture comprising (i) an excess of bromine and (ii) a catalytic quantity of Lewis acid bromination catalyst, and substantially concurrently removing hydrogen bromide coproduct from the reactor in a sufficient amount to form a reaction-derived decabromodiphenyl oxide product of high purity. In general, the duration of the feeding period is inversely related to the temperature at which the refluxing is occurring. In other words, the higher the temperature, the shorter can be the feed time.

As used herein including the claims:

1) The term “reaction-derived” means that the composition of the product is reaction determined and not the result of use of downstream purification techniques, such as recrystallization or chromatography, or like procedures that can affect the chemical composition of the product. Adding water or an aqueous base such as sodium hydroxide to the reaction mixture to inactivate the catalyst, and washing away of non-chemically bound impurities by use of aqueous washes such as with water or dilute aqueous bases are not excluded by the term “reaction-derived”. In other words, the products are directly produced in the synthesis process without use of any subsequent procedure to remove or that removes nonabromodiphenyl oxide from decabromodiphenyl oxide.

2) The term “high purity” means that the reaction-derived DBDPO product comprises more than 99% of DBDPO and nonabromodiphenyl oxide in an amount of less than 1% with, if any, a trace of octabromodiphenyl oxide. Preferably the process forms a reaction-derived product which comprises (i) at least 99.5% of DBDPO and (ii) nonabromodiphenyl oxide in an amount not exceeding 0.5%, preferably not exceeding 0.3%, and still more preferably, not exceeding about 0.1%.

3) The term “substantially continuously” as regards the feeding means that the feeding is either totally continuous with no interruptions whatever or the feeding is interrupted one or more times as long as such interruptions are of short enough duration as not to affect in any significant way the end result of producing a reaction-derived product of high purity.

4) The term “substantially concurrently reducing” as regards the amount of hydrogen bromide means that the reducing is taking place at exactly the same time or at substantially the same time that the feeding is taking place. It is to be clearly understood that the feeding and the reducing of the amount of hydrogen bromide need not start at the same moment in time as there can be a time lag between the commencement of the feed and the evolution of enough hydrogen bromide to initiate the reducing of the amount thereof in the reactor. Likewise, if and when the feeding is terminated, there can be a period of time thereafter during which the amount of hydrogen bromide in the reactor can be reduced. In addition, it should be understood that the term “substantially concurrently reducing” includes one or more interruptions in the removal of hydrogen bromide as long as such interruptions are of short enough duration as not to affect in any significant way the end result of producing a reaction-derived product of high purity.

For the purposes of this invention, unless otherwise indicated, the % values given for DBDPO and nonabromodiphenyl oxide are to be understood as being the area % values that are derived from gas chromatography analysis. A recommended procedure for conducting such analyses is presented hereinafter.

Another embodiment is a process of preparing reaction-derived decabromodiphenyl oxide of high purity, which process comprises maintaining a substantially continuous, inversely related time-temperature feed of diphenyl oxide (DPO) and/or partially brominated DPO to a reactor containing a refluxing reaction mixture comprising an excess of bromine containing Lewis acid bromination catalyst, and substantially concurrently reducing the concentration of hydrogen bromide coproduct dissolved in the liquid phase of the reaction mixture so that a high purity DBDPO product is formed. In this embodiment the higher the bromination reaction temperature, the shorter is the time duration of the feed, and the lower is the pressure in the reactor.

A feature of this invention is that it has been found possible to prepare reaction-derived decabromodiphenyl oxide products containing:

A) (i) at least 99.6% decabromodiphenyl oxide and (ii) nonabromodiphenyl oxide in an amount not exceeding 0.4%;

B) (i) at least 99.7% decabromodiphenyl oxide and (ii) nonabromodiphenyl oxide in an amount not exceeding 0.3%;

C) (i) at least 99.85% decabromodiphenyl oxide and (ii) nonabromodiphenyl oxide in an amount not exceeding 0.15%; and

D) (i) at least 99.95% decabromodiphenyl oxide and (ii) nonabromodiphenyl oxide in an amount of 0.05%.

The above and other embodiments and features of this invention will be still further apparent from the ensuing description, accompanying drawings, and appended claims.

### FURTHER DETAILED DESCRIPTION

As indicated above, the length of the feeding period is temperature dependent. For example when the bromination reaction is taking place at a refluxing temperature in the range of about 57° C. to about 60° C. (about 135° F. to about 140° F.), a feed time of about 4 hours is typically required. On the other hand when the refluxing is occurring at about 77° C. (about 170° F.), a feed time of only about 2 hours is required. In this connection, and without being bound by theory, it appears that this temperature dependence effect is related to the time required to reach the desired equilibrium state described above. Thus, in carrying out a process of this invention, if the temperature-dependent period has not already been determined for the particular operation, interpolation between the above values is a useful guide for a few laboratory experiments for optimization purposes. It is to be noted that at any given temperature use of a higher concentration of catalyst may enable the reaction time to be shortened to some extent, provided that the hydrogen bromide concentration in the liquid phase of the reaction mixture is kept to a minimum or at least low enough as not to prevent preparation of reaction-derived DBDPO of high purity.

Generally speaking, from the viewpoint of productivity and plant throughput, the shorter the feed period used, the better. But pursuant to this invention the feed period used should be sufficiently long at the reaction temperature being used to enable the desired equilibrium state to be reached whereby the reaction-derived product is a high purity product.

Therefore, depending on the temperature at which the bromination is occurring, the feed of DPO and/or partially brominated DPO product(s) should occur during a sufficiently long period in the range of about 2 to about 12 hours, and preferably in the range of about 4 to about 10 hours to reach the desired equilibrium state. When operating at a plant scale this period of time in part represents a compromise between rate of reactor throughput and desire for as slow a feed as is practicable for achieving the desired product purity. Thus, the duration of the substantially continuous feed should be a period of time that is prolonged yet consistent with achieving an economically acceptable plant throughput. The use of a slow feed is desirable as it provides a longer period of time for a given quantity of DPO or partially brominated DPO to reach the decabromodiphenyl oxide stage before significant precipitation of nonabromodiphenyl oxide encased in decabromodiphenyl oxide particles takes place.

In practicing a process of this invention it is important to reduce the content of hydrogen bromide present in the reactor. Among various ways of achieving such reduction in the amount of hydrogen bromide present in the reactor are the following:
A combination of vigorous refluxing of the bromine in the reactor, withdrawal of the hydrogen bromide vapor phase from the reactor, and efficient condensation of bromine vapors being withdrawn with the hydrogen bromide is desirable and is preferably utilized.
Use of a fractionation column to effectively separate as much HBr from the bromine in the column as feasible. In this way the bromine returning to the reactor carries less, if any, HBr back into the reactor. The fractionation column can be a packed column or it can be free of packing, and should be designed to effect an efficient separation of HBr from bromine.
An inert gas purge of the reactor (e.g., with argon, neon, or preferably nitrogen) to carry away HBr is useful.
Use of bromine in the vapor state as a stripping gas. Besides carrying away HBr, the use of bromine vapors is a way of introducing more heat into the reactor and thereby contributing to more vigorous refluxing within the system.
Operation at atmospheric, subatmospheric or superatmospheric pressures to enable a refluxing condition of the reaction mixture at the selected process temperature.
Since the bromination is conducted in excess refluxing bromine, the reactor is of course equipped with a reflux condenser and preferably a reflux fractionation column. This should be designed to return to the reaction as little HBr in the condensed bromine as is technically and economically feasible under the circumstances.

In all cases, the hydrogen bromide leaving the reaction system is preferably recovered for use or sale. Recovery can be achieved by use of a suitable scrubbing system using one or more aqueous liquid scrubbers such as water, or dilute NaOH solution.

The relationship between bromination reaction temperature and pressure under which the bromination is being operated is worthy of comment. Ideally it is desirable to operate at as high a temperature as possible and as low a pressure as possible to adequately reduce the HBr concentration in the bromine, because in this way more HBr is removed from the reactor. Sampling a refluxing bromination reaction mixture of this type in order to assay the percentage of HBr dissolved in the Br₂ at any given time is not deemed feasible when using ordinary laboratory or plant equipment. Such sampling requires special equipment such as built-in stationary probes to periodically remove representative samples of the reaction mixture from the reactor. Thus when using ordinary plant equipment, operation at maximum temperature and minimum pressure is desirable as a way of reducing the HBr concentration in the bromine. However, maintaining a high reaction temperature in such a reaction system is not as easy as it might appear. For one thing, considerable heat input is required to the reaction mixture, and this can impose limitations in existing plant equipment. Consequently, in most cases it is desirable when operating on a commercial scale to conduct the reaction at a mildly elevated pressure (e.g., in the range of about 5 to about 20 psig (ca. 1.36×10⁵ to 2.39×10⁵ Pa)), and having the temperature high enough to effect vigorous refluxing to thereby keep the HBr concentration in the bromine low as more HBr is removed from the reactor.

This invention enables the preparation of highly pure DBDPO products that are derived from the bromination of diphenyl oxide and/or partially brominated diphenyl oxide. For example, it is now possible to prepare reaction-derived decabromodiphenyl oxide of a purity of at least about 99%. Indeed, it is deemed possible to prepare reaction-derived products that contain at least about 99+ % DBDPO and that contain amounts of nonabromodiphenyl oxide not exceeding 0.5%, preferably 0.3% or less, more preferably, no more than about 0.1%, and even more preferably no more than about 0.05%. Such products can be said to be “reaction-derived” since they are reaction determined and not the result of use of downstream purification techniques, such as recrystallization, chromatography, or like procedures. In other words, the products are of high purity.

In the various embodiments of this invention, the feeds to the refluxing bromine-Lewis acid catalyst-containing reaction mixtures can be diphenyl oxide (DPO) itself or one or a mixture of partially brominated diphenyl oxides formed by brominating diphenyl oxide with bromine in the absence of a catalyst. Such individual products and mixtures thereof can be used as feeds in the practice of this invention. The partially brominated DPO, which can be used as the feed in the practice of this invention, typically contains in the range of about 0.5 to about 4 atom(s) of bromine per molecule of DPO. Somewhat higher amounts of uncatalyzed ring-bromination of DPO can be accomplished under pressure, e.g., perhaps up to, say 5 or possibly even 6 atoms of bromine per molecule, by conducting the uncatalyzed reaction under pressure, or by use of a catalyst and such partially brominated DPO products or mixture can be used as feeds in the practice of this invention. In all cases, prior to its use as the feed to the refluxing bromine containing Lewis acid bromination catalyst, the hydrogen bromide coproduct should be removed from the partially brominated DPO feed or at least the amount of residual hydrogen bromide coproduct in the partially brominated DPO should be substantially reduced. The feed of the DPO and/or partially brominated DPO product(s) added to the refluxing reaction mixture is preferably a substantially continuous feed. However, pulsed feeds and variations in the rate of feed are permissible provided that such pulsations or feed rate variations do not result in any significant increase in the amount of hydrogen bromide present in the reaction mixture as compared to the same reaction mixture under the same reaction conditions where a substantially continuous feed rate is used. Thus, the term “substantially continuous” or like term includes pulsed feeds and feeds with such variations in the rate of feed.

The DPO and/or partially brominated DPO can be fed as solids, but preferably the feed is in molten form or as a solution in a solvent such as methylene bromide or bromoform. To prevent freeze up in the feed conduit, DPO is desirably fed at a temperature of in the range at least of 28 to 35° C. Higher temperatures can be used if desired.

Excess bromine is used in the Lewis acid catalyzed bromination reaction. Typically, the reaction mixture will contain in the range of at least about 14 moles of bromine per mole of DPO to be fed thereto, and preferably, the reaction mixture contains in the range of about 16 to about 25 moles of bromine per mole of DPO to be fed thereto. It is possible to use more than 25 moles bromine per mole of DPO but this offers no advantage. When the feed is partially brominated DPO, enough bromine should be present to provide in the range of about 4 to about 12 moles of excess bromine over the amount required to perbrominate the partially brominated DPO. When the feed is a mixture of DPO and partially brominated DPO, the amount of excess bromine should be enough to provide a corresponding excess over the amounts sufficient to perbrominate the DPO and the partially brominated DPO.

Typically the refluxing temperature of bromine at atmospheric or slightly elevated pressures is in the range of about 57 to about 59° C. but when operating at higher elevated pressures somewhat higher temperatures are used in order to maintain a refluxing condition.

If desired, a suitable solvent can be included in the reaction mixtures. This can be advantageous in that one can have a higher reaction temperature and possibly a lower HBr concentration in the bromine thereby giving higher purity DBDPO. Among such solvents are methylene bromide and bromoform.

Various iron and/or aluminum Lewis acids can be added to the bromine to serve as the bromination catalyst. These include the metals themselves such as iron powder, aluminum foil, or aluminum powder, or mixtures thereof. Preferably use is made of such catalyst materials as, for example, ferric chloride, ferric bromide, aluminum chloride, aluminum bromide, or mixtures of two or more such materials. More preferred are aluminum chloride and aluminum bromide with addition of aluminum chloride being more preferred from an economic standpoint. It is possible that the makeup of the catalyst may change when contained in a liquid phase of refluxing bromine. For example, one or more of the chlorine atoms of the aluminum chloride may possibly be replaced by bromine atoms. Other chemical changes are also possible. The Lewis acid should be employed in an amount sufficient to effect a catalytic effect upon the bromination reaction being conducted. Typically, the amount of Lewis acid used will be in the range of about 0.06 to about 2 wt %, and preferably in the range of about 0.2 to about 0.7 wt % based on the weight of the bromine being used.

After all DPO and/or partially brominated DPO is added, the reaction mixture can be kept at reflux for a suitable period of time to ensure completion of the perbromination to DPDPO. A period of up to about one hour can be used. Generally speaking, the benefits of such post-reaction refluxing tend to offset by the prolongation of the overall batch operation, and thus use of such post reflux, though permissible, is not preferred.

Termination of the bromination reaction is typically effected by deactivating the catalyst with water and/or an aqueous base such as a solution of sodium hydroxide or potassium hydroxide.

### Recommended Gas Chromatographic Procedure

The gas chromatography is on a Hewlett-Packard 5890 gas chromatograph using a 12QC5 HTS capillary column, 12 meter, 0.15μ film thickness, 0.53 mm diameter, part number 054657, SGE, Inc. (2007 Kramer Lane, Austin, Tex. 78758). Conditions were: 1:10 split injection, column head pressure 9 psig (ca. 1.63×10⁵ Pa), injector temperature 325° C., flame ionization detector temperature 350° C., and column temperature 300° C. isothermal. The carrier gas was helium. Samples were prepared by dissolving, with warming, 0.05 grams in 10 mL of dibromomethane and injection of 1 microliter of this solution. The integration of the peaks was carried out using Target Chromatography Analysis Software from Thru-Put Systems, Inc. (5750 Major Blvd., Suite 200, Orlando Fla. 32819; currently owned by Thermo Lab Systems). However, other and commercially available software suitable for use in integrating the peaks of a chromatograph may be used.

The GC procedure described above provides a trace having several peaks. The first peak is deemed to be the meta- and para-hydrogen isomers of nonabromodiphenyl oxide. The second peak is deemed to be the ortho-hydrogen isomer of nonabromodiphenyl oxide. The main peak, of course, is decabromodiphenyl oxide.

The practice of embodiments of the invention and advantages achievable by practice of the embodiments of the invention are illustrated in the following Examples. These Examples are not intended to impose limitations on the overall scope of the invention.

### EXAMPLE 1

### Catalyzed Bromination

A reactor was configured from a 1-liter Morton flask with a mechanical stirrer, thermometer, 60 mL addition funnel, and fractionation column (10″×1″ with 5 mm×5 mm Raschig rings) topped by a 0° C. reflux condenser. The outlet of the condenser was connected to a H₂O trap. A small N₂ purge was added to the line from the condenser to the H₂O trap. The reactor was charged with 3.5 g of AlCl₃ and 1577 g of bromine (containing 11 ppm H₂O). The addition funnel was charged with 47.04 g of diphenyl oxide. The reactor was heated to 55° C. and the diphenyl oxide was added drop-wise supersurface. The time for the initiation of the diphenyl oxide addition was noted. The reactor was heated by a mantle. Twenty-seven minutes into the diphenyl oxide feed, half of the diphenyl oxide had been added and the reaction mass temperature was 56° C. One and a quarter hours after the diphenyl oxide feed was initiated, all of the diphenyl oxide had been added and the reaction mass temperature was 57° C. The compressor on the refrigeration system was shut off to allow slow warm-up of the condenser. The reaction mass was refluxed through the fractionation column. At one hour and 18 minutes after feed initiation, the reaction mass temperature was 59° C. Two hours and three minutes after diphenyl oxide feed initiation the condenser water was at 20° C. and the reaction temperature was at 61° C. At two hours and seven minutes after feed initiation, the condenser water was at 30° C. Thirty two minutes later the addition funnel was replaced with a N₂ inlet. A slow N₂ purge of the reactor was started. The reaction mass temperature was 61° C. The N₂ purge was at 100 mL/min into the vapor space of the reactor. Four hours and fifty minutes after the initiation of the diphenyl oxide feed, the reaction mass temperature was 61° C. and the condenser water was at 37° C. At six hours and 15 minutes after the initiation of the diphenyl oxide feed the reaction mass was cooled to 55° C., 350 mL deionized H₂O was added, the fractionation column was removed, and the reactor was set for distillation. Br₂ was distilled off. When most of the Br₂ was gone 150 mL more deionized water was added. The remaining Br₂ was distilled off to 100° C. The remaining mixture was cooled to 60° C., and 30 mL of 25% NaOH was added to pH 13-14. The resultant mix was filtered and washed well with deionized water. A sample was subjected to GC analysis. The GC trace showed the product to contain 0.21% of the first nonabromodiphenyl oxide peak (deemed to be meta- and para-hydrogen isomers), 0.24% of the second nonabromodiphenyl oxide peak (deemed to be the ortho-hydrogen isomer) and 99.54% of decabromodiphenyl oxide. The sample was oven dried.

### EXAMPLE 2

### Preparation of Partially Brominated DPO

In a 500 mL 4-neck flask equipped with condenser, addition funnel, thermometer, and ⅛″ diptube for take off was placed 437 g of bromine. To this was added 48.6 g of diphenyl oxide under reflux. All of the diphenyl oxide was added in 45 minutes. 120 g more bromine was added and the solution was refluxed at 63° C. under a 0° C. condenser for 30 minutes.

### Catalyzed Bromination

A 1-liter 4-neck flask equipped with mechanical stirrer, a 1/16″ O.D. ( 1/32″ ID) dip tube, thermometer, a 9″×1″ fractionation column (5 mm×5 mm Raschig ring) and topped by a 0° C. condenser, were charged 4.03 g of AlCl₃ and 746 g of bromine. This mixture was brought to reflux and the above previously-prepared mixture of partially brominated DPO was pumped in at ≈1 mL/min. The resultant reaction mass was kept at reflux through the fractionation column with stirring at 200-300 rpm. The pump was a peristaltic pump using 0.8 mm I.D. Viton® tubing in the pump. Reaction mass temperature was 59° C. At two hours and fifty six minutes after the initiation of the solution feed, the 500 mL flask was empty. 15 g of bromine was added to a 500 mL flask and this bromine was also pumped into the 1-liter flask. Nitrogen at ≈100 mL/min, was then fed down the diptube (subsurface) as the reaction mass was refluxed one hour longer. 500 mL of deionized water was then added, the fractionation column was removed and the reactor was set to distill bromine from the reaction mass. Bromine was distilled to 100° C. and the remaining reaction mass was cooled to 60° C. 8 g of NaOH in 40 mL of water was added (pH 13-14). The resultant mixture was filtered and H₂O washed giving a washed product. GC analysis showed the product to contain 0.058% of the first of nonabromodiphenyl oxide peak (meta and para isomers) and 0.085% of the second nonabromodiphenyl oxide peak (ortho isomer). After oven drying overnight at 110° C., the dried product weighed 265 g.

### EXAMPLE 3

### Preparation of Partially Brominated DPO

To the 500 mL 4-neck flask equipped as in Example 2 was added 732 g of Br₂. Molten diphenyl oxide (49.3 g) was added dropwise at bromine reflux over 40 minutes. The solution was refluxed (0° C. condenser) 45 minutes longer.

### Catalyzed Bromination

To the 1-liter flask, equipped as in Example 2, were added 4.0 g of AlCl₃ and 595 g of bromine. This mixture was brought to reflux through the fractionation column and the contents of the 500 mL flask were pumped in at ˜0.5 mL/min via the 1/16″ OD ( 1/32″ ID) diptube subsurface to the resulting reaction mass. The reaction mass temperature was 59° C. The reaction mass was kept at hard reflux throughout the solution addition. The temperature of the cooling water on condenser was 17° C. Eight hours later, all of the contents of the 500 mL flask had been pumped into the 1-liter flask. About 5 mL of bromine was added to the 500 mL flask and this bromine was pumped into the 1-liter flask. The reaction mass was then refluxed 15 minutes longer with a N₂ purge (about 100-200 mL/min. down the diptube). The reaction mass was cooled partially, and 500 mL of H₂O was added and the reactor was set for distillation. Bromine was distilled to 100° C. and the reaction mass was cooled to 60° C. Excess 5% NaOH added to pH 12. The solid product was collected and washed well with H₂O. A sample was analyzed by GC. GC analysis showed the product to contain 0.017% of the first nonabromodiphenyl oxide peak (meta and para isomers), 0.031% of the second nonabromodiphenyl oxide peak (ortho isomer) and 99.95% of decabromodiphenyl oxide. The remainder of the product was dried overnight at 130° C. and, after drying, weighed 263 g.

### EXAMPLE 4

### Preparation of Partially Brominated DPO

To 50.0 g of DPO in a 500 mL flask equipped with magnetic stirrer, addition funnel, thermometer, and condenser was added 29 g of bromine dropwise over 30 minutes. The reactor was cooled in a water bath. When all the bromine had been added, the reactor was purged with nitrogen to facilitate pumping and prevent HBr breakout in the line.

### Catalyzed Bromination

In a 1-liter 4-neck Morton flask were placed 4.05 g of AlCl₃ and 1230 g of bromine. The flask was equipped with 1/32-inch I.D. diptube (subsurface) and a fractionation column as described in Example 2 and topped with a tap water cooled Friedrich condenser. This mixture was brought to reflux and the DPO mixture was pumped into the reactor via the 1/32-inch diptube. Reaction temperature was 59° C. Heating was such that bromine vapor rose to about ½ of the height of the condenser before totally condensing. The temperature of the cooling water exiting the condenser was 25.6° C. After 6 hours and 58 minutes of feeding, all of the DPO had been added except for about 1 g remaining in the flask. The diptube was removed and the reaction mixture was refluxed 10 minutes longer. Water (500 mL) was added and the reactor was set for bromine distillation. Bromine was distilled to a reaction temperature of 100° C. 658 g of bromine was collected. The reactor was cooled, 5% NaOH was added to pH of about 13, and the product was collected and washed. GC analysis showed the product to contain 0.020% of the first nonabromodiphenyl oxide peak (meta and para isomers), 0.080% of the second nonabromodiphenyl oxide peak (ortho isomer) and 99.90% of decabromodiphenyl oxide. The product was dried overnight at 130° C. After drying overnight the product weighed 265.2 g.

### EXAMPLE 5

### Preparation of Partially Brominated DPO

To the 500 mL flask (equipped as described in Example 4) containing 51.67 g of DPO was added 30.0 g of bromine over about 10 minutes with cooling in a water bath. After stirring for 15 minutes this mixture was nitrogen sparged to remove HBr.

### Catalyzed Bromination

In a 1-liter jacketed 4-neck flask equipped with a thermocouple, 1/32-inch I.D. dip tube, mechanical stirrer and 9 inch×1 inch column (packed with 5 mm×5 mm Raschig rings) and topped with a water-cooled Friedrich condenser, and heated by circulating hot water through the jacket, the water temperature being controlled to give the desire reaction temperature, were placed 4.0 g of AlCl₃ and 1240 g of bromine. This mixture was heated to 56.7° C. and DPO addition initiated. After a DPO feed time of 9 hours and 42 minutes, during which time the reaction temperature was maintained mainly at 56.1° C. to 57.1° C., the feed was stopped and the mixture was refluxed 5 minutes longer under a nitrogen purge of the reaction flask. Then 500 mL of water was added and the flask was set for distillation. 617 Grams of bromine was distilled to a temperature of 80° C. The stirrer in the flask was stopped and the resultant water phase was decanted. The solids were washed with 500 mL of water and the water was decanted. Then 500 mL of water and 10 g of NaOH were added to the solids and the mixture was stirred well. Then the mixture was filtered and washed well on the filter leaving the product as a filter cake. A sample after drying was shown by GC analysis to contain 0.031% of the first nonabromodiphenyl oxide peak (meta and para isomers), 0.205% of the second nonabromodiphenyl oxide peak (ortho isomer) and 99.764% of decabromodiphenyl oxide. The product was oven dried and weighed 267.2 g.

### EXAMPLE 6

### Preparation of Partially Brominated DPO

To the 500 mL flask (equipped as described in Example 4) containing 47.3 g of DPO was added over about 10 minutes, 28.0 g of bromine with stirring and cooling at room temperature.

### Catalyzed Bromination

In a 1-liter jacketed flask equipped with mechanical stirrer, Friedrich condenser (water cooled at about 25° C.), and with a 1/32-inch diptube but without a fractionation column were placed 3.8 g of AlCl₃ and 885 g of bromine. After a feed time of about 7.25 hours, all of the partially brominated DPO had been fed from the flask. The reaction temperature was maintained at 56.3° C. to 57.2° C. throughout the addition. The reaction mixture was refluxed for 4 minutes as the temperature rose to 58.4° C., then 450 mL of water was added and the reactor was set for distillation. The product was distilled to 170° F. (about 77° C.) and 312 g of bromine was collected. The water layer was decanted from the reactor, 400 mL of water was added, stirred, and discarded. Then 400 mL of water and 10 g of NaOH were added, the mixture was stirred well and product was collected and water washed on a filter. GC analysis showed the product was composed of 99.71% of decabromodiphenyl oxide, and 0.034 and 0.259% of the first and second nonabromodiphenyl oxide isomers, respectively. The product was placed in a 125° C. oven and after drying overnight weighed 252.0 g.

### EXAMPLE 7

### Preparation of Partially Brominated DPO

To the 500 mL flask (equipped as described in Example 4) containing 49.1 g of DPO was added over about 10 minutes, 29.7 g of bromine. This was purged with nitrogen to remove HBr.

### Catalyzed Bromination

In a 1-liter jacketed flask equipped as in Example 6 (no fractionation column) were placed 3.8 g of AlCl₃ and 884 g of bromine. The mixture was heated to 59° C. and a feed of the partially brominated DPO formed above was initiated. The feed through the 1/32-inch diptube was set at a rate of about 0.21 mL per minute. All the partially brominated DPO was added over 3 hours and 23 minutes, The reaction mixture had been maintained at a temperature of 56.1° C. to 57.1° C. throughout the addition time reflux was continued for about 10 more minutes as the temperature rose to 59.6° C. Then 450 mL of water was added to the reaction mixture and the reactor was set for distillation. The mixture was distilled to 77° C. and 294.5 g of bromine was collected. The mixture was worked up as in Example 6. GC analysis of the product showed 99.59% of decabromodiphenyl oxide and 0.11% and 0.296% of first and second nonabromodiphenyl oxide peaks, respectively. Present in the product were a few “lumps”. One was removed and a GC showed it contained 99.61% of decabromodiphenyl oxide and 0.100 and 0.291% of the first and second nonabromodiphenyl oxide isomers, respectively. The product was oven dried.

### EXAMPLE 8

### Preparation of Partially Brominated DPO

To the 500 mL flask (equipped as in Example 4) containing 49.00 g of DPO was added 31.4 g of bromine over about 10 minutes. Then the mixture was purged with nitrogen.

### Catalyzed Bromination

In a 1-liter jacketed flask, equipped as in Example 6, were placed 3.82 g of AlCl₃ and 988 g of bromine The mixture was heated to 56.0° C. and addition of the partially brominated DPO begun at a feed rate of about 0.18 mL per minute. This feed was maintained for a period of about 4 hours with the temperature fluctuating between 53.0° C. and 54.0° C. The mixture was allowed to reflux for about another 7 minutes with the temperature reaching about 60° C. Then, 450 mL of water was added to the reaction mixture and the reaction vessel was set for distilling bromine. The distillation was conducted to 77° C. whereby an amount of 400.2 g of bromine was recovered. Product was isolated as in Example 5 and oven dried. GC analysis showed 0.093% and 0.471% of the first and second nonabromodiphenyl oxide peaks, respectively, and 99.436% of decabromodiphenyl oxide. After drying over the weekend the product weighed 260.1 g.

### EXAMPLE 9

### Preparation of Partially Brominated DPO

A batch of partially brominated DPO was prepared by slowly adding 131 g of bromine to 236 g of DPO while cooling in a water bath so that the temperature did not exceed 40° C. The resultant partially brominated DPO contained about 0.59 atom of bromine per molecule of DPO.

### Catalyzed Bromination

In a 1-liter jacketed reactor equipped with 0° C. Friedrich condenser, mechanical stirrer, thermocouple well, and 1/32-inch I.D. diptube was placed 4.0 g AlCl₃ and 1021 g of bromine. To this was fed 64.3 g of a portion of the above batch of partially brominated DPO. The feed rate was approximately 0.17 mL per minute with the condenser water set at +1° C. The feed occurred over a period of 4 hours and 17 minutes during which time the reaction temperature was maintained at 56.5 to 56.9° C. The mixture was refluxed for an additional period of about 5 minutes as the temperature rose to 59° C. and then 450 mL of water was added to it. Bromine was distilled off from the reaction mixture up to a temperature of ca 80° C. The amount of bromine collected was 502 g. Water was then decanted from the reaction mixture, and the remainder of the reaction mixture was stirred in 400 mL of water and this wash water was decanted. Then 500 mL of 2% NaOH was added. Product was collected and water washed. GC analysis showed 0.067% and 0.233% of the first and second nonabromodiphenyl oxide isomers, respectively, the second being the ortho isomer, and 99.70% of decabromodiphenyl oxide.

### EXAMPLE 10

### Catalyzed Bromination

Another portion of the same partially brominated DPO as used in Example 9 was used as the feed in this Example. The reactor was a 500 mL pressure reactor (Ace glass #6438-17) with jacket, equipped with a mechanical stirrer, a 1/32-inch diptube to feed the partially brominated diphenyl oxide, ¼-inch O.D. diptube for takeoff, and a 9 inch×¾-inch I.D. uninsulated fractionation column packed with 5 mm×5 mm Raschig rings. The column was fitted at the top with a 0° C. condenser. The condenser was connected to a backpressure regulator to maintain the desire pressure. To this reactor were changed 3.9 g of AlCl₃ and 940 g of bromine. A 90 mL pressure bottle was charged with 59.8 g of the above partially brominated diphenyl oxide having about 0.6 bromine atom per molecule of diphenyl oxide. The pressure bottle was equalized with reactor via a nitrogen purge that entered the system at a back pressure regulator. The contents of the 500 mL pressure reactor were heated to a refluxing temperature of 78.4° C. at a pressure of 12.1 psig (ca. 1.85×10⁵ Pa). Then a feed of the partially brominated DPO was maintained at a flow rate of about 0.17 mL per minute for an overall period of about 3 hours and 39 minutes. The reaction mixture was maintained at a temperature of 74.3° C. to 76.2° C. and at 12.1 to 13.5 psig (ca. 1.85×10⁵ to 1.94×10⁵ Pa) pressure. Operational difficulties in the system were encountered during this time. Nevertheless, 100 mL of water was then charged to the reactor and the reaction mixture was poured into a 1-liter flask. The reaction mixture was stripped of bromine up to a temperature of 100° C. Solids remaining in the reactor were treated with dilute sodium hydroxide solution and combined with the product that had been stripped. This mixture was filtered and washed giving a product containing 0.034% and 0.526% of the first and second nonabromodiphenyl oxide isomers, respectively, the second being the ortho isomer, and 99.44% of DBDPO.

### EXAMPLE 11

### Catalyzed Bromination

Into a 1-liter Morton flask equipped with a Vigreux column (10-inches×1-inch), a 0-5° C. cooled Friedrich condenser, a 1/32-inch diptube, a mechanical stirrer, heating mantle, and a thermocouple well and thermocouple were placed 6.9 g of AlCl₃ and 909 g of bromine. This was brought to a hard reflux (90 volts of heating mantle) as a total of 56.5 g of another portion of the same partially brominated DPO as used in Example 9 was pumped into this reactor at a rate of 0.4 mL per minute. The reaction was maintained mainly at 58.5° C. to 59.4° C. with one excursion at the beginning to 56.9° C. Reflux was ⅔ up the Friedrich condenser during the addition, which occurred over 100 minutes. The mixture was refluxed 10 more minutes, during which time the reaction temperature rose to 60.4° C. The reaction mixture was then cooled in a water bath to 40° C. and 500 mL of tap water was added to the reaction mixture. The Vigreux column was removed and the reactor set for distillation. Bromine was stripped from the reaction mixture up to 100° C., and a total of 430 g of bromine was recovered. The reaction mixture was cooled to 60° C. and 25% aqueous caustic solution was added to a pH of 13. The solid product was collected and water washed and then oven dried. GC of a sample of the product showed 0.084% of the first nonabromodiphenyl oxide isomer (a mixture of meta and para hydrogen containing isomers) and 0.220% of a second nonabromodiphenyl oxide isomer (ortho-hydrogen isomer) and 99.696% of decabromodiphenyl oxide. After oven drying for 2 hours the product weighed 242.8 g.

### EXAMPLE 12

### Catalyzed Bromination

Example 11 was reproduced as exactly as possible using the same equipment, except less catalyst was used. Charged to the reactor were 3.00 g of AlCl₃ and 913 g of bromine. 56.5 g of the same partially brominated DPO as used in Example 9 was pumped in over 101 minutes as the temperature was maintained at 58.3° C. to 59.1° C. Heating via the heating mantle using the same voltage setting as in Example 11. It was heated at reflux 10 minutes longer as the temperature rose to 60.4° C. The product was isolated in the same manner as in Example 11, 431 grams bromine being recovered. GC analysis of the product showed 0.185% of a first nonabromodiphenyl oxide isomer (believed to be a mixture of meta and para hydrogen containing isomers) and 0.181% of a second nonabromodiphenyl oxide (believed to be ortho-hydrogen isomer) and 99.634% of decabromodiphenyl oxide. After oven drying for 2 hours the product weighed 246.0 g.

From a comparison of the results of Examples 11 and 12, it appears that, at least on a laboratory scale of operation, a 59° C. a kinetic limitation arises as evidenced by the meta- and para-hydrogen-containing nonabromodiphenyl oxide isomers being significantly less (0.084 versus 0.185) when a higher level of aluminum chloride catalyst was used. Typical for a 4-hour feed time and the lower level of aluminum chloride catalyst is the formation of about 0.07% of the meta- and para-hydrogen-containing nonabromodiphenyl oxide isomers.

The Drawings show illustrative GC traces formed using the recommended gas chromatographic procedure described hereinabove. In these traces, the abscissa is time in minutes and the ordinate is the detector response.

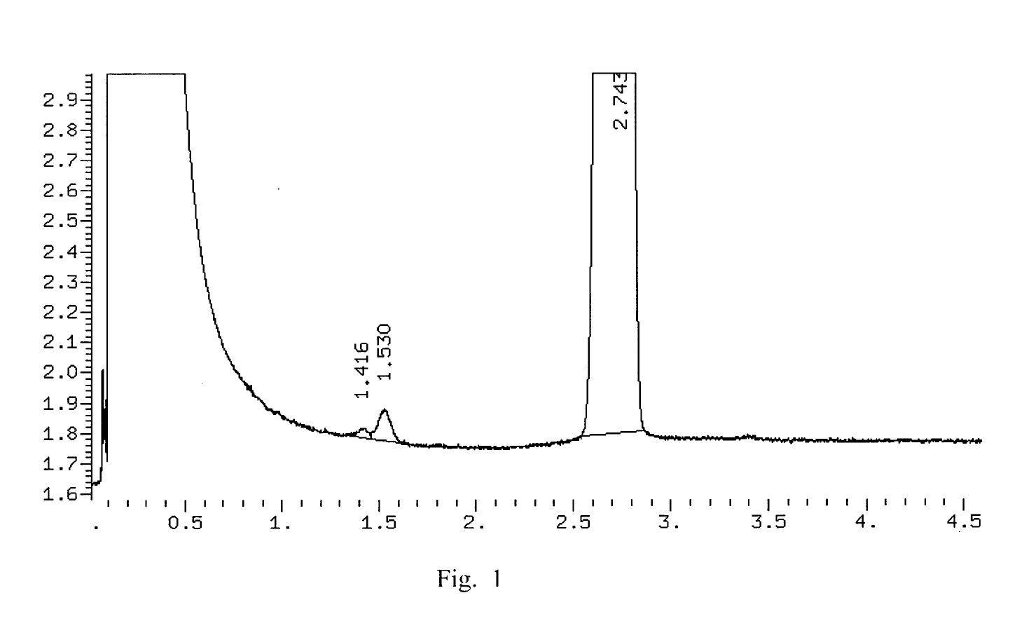

A copy of the GC trace of the product formed in Example 4 appears as FIG. 1. In FIG. 1, the peak at 1.416 represents the area percentage of the isomer deemed to be the meta- para-isomer of nonabromodiphenyl oxide whereas the peak at 1.530 represents the area percentage of the isomer deemed to be the ortho-isomer of nonabromodiphenyl oxide. The peak at 2.743 represents the area percentage of decabromodiphenyl oxide.

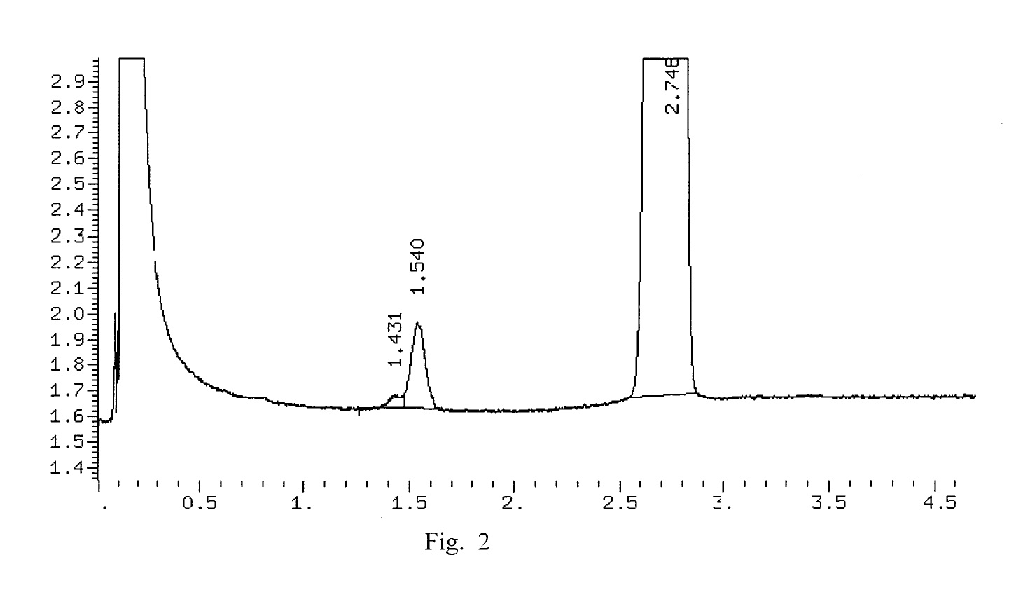

FIG. 2 is a copy of the GC trace of the product formed in Example 6. In the trace of FIG. 2, the peak at 1.431 represents the area percentage of the isomer deemed to be the meta-para-isomer of nonabromodiphenyl oxide. The peak at 1.530 represents the area percentage of the isomer deemed to be the ortho-isomer of nonabromodiphenyl oxide. The large peak between about 2.5 and about 2.9 labeled “BR10” represents the area percentage of decabromodiphenyl oxide.

The DBDPO products formed in processes of this invention are white or slightly off-white in color. White color is advantageous as it simplifies the end-users task of insuring consistency of color in the articles that are flame retarded with the DBDPO products.

The DBDPO products formed in the processes of this invention maybe used as flame retardants in formulations with virtually any flammable material. The material may be macromolecular, for example, a cellulosic material or a polymer. Illustrative polymers are: olefin polymers, cross-linked and otherwise, for example homopolymers of ethylene, propylene, and butylene; copolymers of two or more of such alkene monomers and copolymers of one or more of such alkene monomers and other copolymerizable monomers, for example, ethylene/propylene copolymers, ethylene/ethyl acrylate copolymers and ethylene/propylene copolymers, ethylene/acrylate copolymers and ethylene/vinyl acetate copolymers; polymers of olefinically unsaturated monomers, for example, polystyrene, e.g. high impact polystyrene, and styrene copolymers, polyurethanes; polyamides; polyimides; polycarbonates; polyethers; acrylic resins; polyesters, especially poly(ethyleneterephthalate) and poly(butyleneterephthalate); polyvinyl chloride; thermosets, for example, epoxy resins; elastomers, for example, butadiene/styrene copolymers and butadiene/acrylonitrile copolymers; terpolymers of acrylonitrile, butadiene and styrene; natural rubber; butyl rubber and polysiloxanes. The polymer may be, where appropriate, cross-linked by chemical means or by irradiation. The DBDPO products of this invention can be used in textile applications, such as in latex-based back coatings.

The amount of a DBDPO product of this invention used in a formulation will be that quantity needed to obtain the flame retardancy sought. It will be apparent to those skilled in the art that for all cases no single precise value for the proportion of the product in the formulation can be given, since this proportion will vary with the particular flammable material, the presence of other additives and the degree of flame retardancy sought in any give application. Further, the proportion necessary to achieve a given flame retardancy in a particular formulation will depend upon the shape of the article into which the formulation is to be made, for example, electrical insulation, tubing, electronic cabinets and film will each behave differently. In general, however, the formulation, and resultant product, may contain from about 1 to about 30 wt %, preferably from about 5 to about 25 wt % DBDPO product of this invention. Masterbatches of polymer containing DBDPO, which are blended with additional amounts of substrate polymer, typically contain even higher concentrations of DBDPO, e.g., up to 50 wt % or more.

It is advantageous to use the DBDPO products of this invention in combination with antimony-based synergists, e.g. Sb₂O₃. Such use is conventionally practiced in all DBDPO applications. Generally, the DBDPO products of this invention will be used with the antimony based synergists in a weight ratio ranging from about 1:1 to 7:1, and preferably of from about 2:1 to about 4:1.

Any of several conventional additives used in thermoplastic formulations may be used, in their respective conventional amounts, with the DBDPO products of this invention, e.g., plasticizers, antioxidants, fillers, pigments, UV stabilizers, etc.

Thermoplastic articles formed from formulations containing a thermoplastic polymer and DBDPO product of this invention can be produced conventionally, e.g., by injection molding, extrusion molding, compression molding, and the like. Blow molding may also be appropriate in certain cases.

Components referred to by chemical name or formula anywhere in the specification or claims hereof, whether referred to in the singular or plural, are identified as they exist prior to coming into contact with another substance referred to by chemical name or chemical type (e.g., another component, a solvent, or etc.). It matters not what chemical changes, transformations and/or reactions, if any, take place in the resulting mixture or solution as such changes, transformations, and/or reactions are the natural result of bringing the specified components together under the conditions called for pursuant to this disclosure. Thus the components are identified as ingredients to be brought together in connection with performing a desired operation or in forming a desired composition. Also, even though the claims hereinafter may refer to substances, components and/or ingredients in the present tense (“comprises ”, “is”, etc.), the reference is to the substance, component or ingredient as it existed at the time just before it was first contacted, blended or mixed with one or more other substances, components and/or ingredients in accordance with the present disclosure. The fact that a substance, component or ingredient may have lost its original identity through a chemical reaction or transformation during the course of contacting, blending or mixing operations, if conducted in accordance with this disclosure and with ordinary skill of a chemist, is thus of no practical concern.

Each and every patent or publication referred to in any portion of this specification is incorporated in toto into this disclosure by reference, as if fully set forth herein.

That which is claimed is:

1. A process of preparing reaction-derived decabromodiphenyl oxide product of high purity, which process comprises feeding diphenyl oxide and/or partially brominated diphenyl oxide substantially continuously over a period in the range of about 2 to about 12 hours into a reactor containing a refluxing reaction mixture comprising (i) an excess of bromine and (ii) a catalytic quantity of Lewis acid bromination catalyst, and substantially concurrently removing hydrogen bromide coproduct from the reactor in a sufficient amount to form a reaction-derived decabromodiphenyl oxide product of high purity.

2. A process as in claim 1 wherein a flow of inert gas is passed through the reactor during the feed and/or upon completion of the feed.

3. A process as in claim 1 wherein a flow of bromine vapor is passed through the reactor during the feed and/or upon completion of the feed.

4. A process as in claim 1 wherein the reaction mixture is subjected to fractionation in a fractionation column during the feed and/or upon completion of the feed.

5. A process as in claim 1 wherein the period utilized is inversely related to the temperature at which the refluxing is occurring.

6. A process as in claim 1 wherein the reaction mixture is maintained at about atmospheric pressure.

7. A process as in claim 1 wherein said high purity decabromodiphenyl oxide product contains (i) at least 99.5% decabromodiphenyl oxide and (ii) nonabromodiphenyl oxide in an amount not exceeding 0.5%.

8. A process of preparing reaction-derived decabromodiphenyl oxide of high purity, which process comprises maintaining a substantially continuous, inversely related time-temperature feed of diphenyl oxide and/or partially brominated diphenyl oxide to a reactor containing a refluxing reaction mixture comprising an excess of bromine containing Lewis acid bromination catalyst, and substantially concurrently reducing the concentration of hydrogen bromide coproduct dissolved in the liquid phase of the reaction mixture so that a high purity decabromodiphenyl oxide product is formed.

9. A flammable macromolecular material containing a flame retardant amount of a reaction-derived product of claim 8.

10. A material as in claim 9 wherein the macromolecular material is a thermoplastic polymer, a thermoset polymer, or a latex-based coating.
